(19) United States
(12) Patent Application Publication
(10) Pub. No.: US 2007/0098767 A1
(43) Pub. Date: May 3, 2007

(54) Substrate and personal-care appliance for health, hygiene, and/or environmental applications(s); and method of making said substrate and personal-care appliance

(76) Inventors: Kenneth B. Close, New London, WI (US); Jonathan K. Arendt, Appleton, WI (US); Grey V. Anderson, Larsen, WI (US); Thomas Vercauteren, Greenville, WI (US); Wael R. Joseph, Appleton, WI (US); Bhaskara C. Chilakamarri, Appleton, WI (US)

Correspondence Address: KIMBERLY-CLARK WORLDWIDE, INC., 401 NORTH LAKE STREET, NEENAH, WI, 54956, US

(21) Appl. No.: 11/265,390

(22) Filed: Nov. 1, 2005

(51) Int. Cl. A61K 9/70

(52) U.S. Cl. 424/443

(57) ABSTRACT

Substrates, and personal-care appliances made from such substrates, include an inter-bonded fibrous layer having shaped discontinuities and reinforcing strands attached to said inter-bonded fibrous layer. Pores between the fibers in the inter-bonded fibrous layer are suited to help hold liquid. The shaped discontinuities are suited to help generate suds or lather should the substrate or personal-care appliance be used in combination with a cleaning composition, soap formulation, or other such material having a surface-active agent or other chemical or compound that helps generate lather. The reinforcing strands help strengthen, or improve the wet resilience of, the inter-bonded fibrous layer. Furthermore, by selecting the appropriate ingredients of the reinforcing strands, the strands may be stiffer than the inter-bonded fibrous layer, thereby helping to provide some exfoliating character to the substrate or personal-care appliance.

People use various substrates, and personal-care appliances made from such substrates, for a number of health, hygiene, and/or environmental applications.

Bathing poufs, one example of a personal-care appliance, are popular bathing devices generally made from netted or meshed substrates. Bathing poufs may be used in place of or in conjunction with wash cloths during a shower or bath. Bathing poufs may be used with liquid or bar soap or other such formulations to create lather during bathing, thereby providing mechanical cleansing and/or exfoliation benefits not provided by soap alone. Moisturizing compositions, or soaps containing such compositions, are frequently applied with bathing poufs. Such bathing poufs may also be used without soap, thereby providing mechanical scrubbing and/or exfoliation benefits.

Substrates and personal-care appliances used for health, hygiene, and/or environmental applications, such as the aforementioned pouf, may implicate potential technical problems or disadvantages. One possible problem involves the formation of mildew or other biological-growths on the personal-care appliance. For example, such growths may form on the netted material of a pouf and/or any cord attached to the netted material. This is especially true near the center of the pouf where the netted material tends to be more concentrated, thus more easily retaining or trapping water, soap, and other matter (e.g., hair, dirt, dead skin cells, fecal material, and/or other bodily waste). As a result, germs, microbes, viruses, bacteria, molds, mildews, and fungi may build up on the pouf, especially at the pouf's interior. Due to the configuration of bathing poufs, it is difficult to determine if the poufs have been adequately cleaned or rinsed, or if mildew or molds are present. Once the growth of mildew and molds has been detected, it may be difficult to clean the bathing poufs to eliminate the mildew and molds. Such biological growths may occur in other personal-care appliances, such as wash cloths or similar substrates.

Another potential technical problem with substrates, and personal-care appliances made from such substrates (e.g., poufs made from a plastic netting, wash cloths, etc.), is that it can be difficult to balance various characteristics desired by a user of the appliance. For example, for certain health, hygiene, and/or environmental applications, such as bodily cleansing, some users may want the personal-care appliance to be soft. Other users may want the same appliance to help exfoliate skin; i.e., they want the appliance to be relatively stiff. Thus the personal-care appliance may need to balance two seemingly contradictory properties: softness/flexibility (for a user's comfort) versus stiffness/rigidity (to help exfoliate a user's skin). Another pair of seemingly contradictory properties relates to a personal-care appliance's ability to hold liquid versus its ability to generate bubbles. Generally, a substrate with less open area (which can translate to smaller pores throughout an appliance comprising the substrate) will hold more liquid. To generate bubbles with a soap or surfactant formulation, however, more open area is typically needed. Again a personal-care appliance, such as the pouf discussed above, may have to balance two characteristics that are seemingly mutually exclusive of one another: good water-holding capacity (less open area in substrate) versus good bubble-generating ability (more open area in substrate).

What is needed is a substrate, and a personal-care appliance comprising said substrate, that are capable of: being made such that the substrate and appliance are for limited use—i.e., they are disposable (e.g., disposed of after less than about 10 individual uses; more suitably less than about 5 individual uses), thereby helping reduce the chances of undesirable accumulations of unhealthy microorganisms on the substrate and/or personal-care appliance; and/or balancing pairs of properties that may be somewhat contradictory (e.g., softness/flexibility versus stiffness/rigidity; good liquid-holding capacity versus good bubble-generating ability). Furthermore, processes for making said substrate, and personal-care appliances comprising said substrate, are needed.

### SUMMARY

We have found that a substrate comprising an interbonded fibrous layer having shaped discontinuities (e.g., circular holes or depressions in the fibrous layer that are significantly larger than the pores defined by the interbonded fiber, with the holes or depressions optionally being contiguous with projections emanating from the surface of the interbonded fibrous layer) and reinforcing strands (i.e., strands that are generally larger and stiffer than fiber making up the interbonded fibrous layer) attached to said fibrous layer, and personal-care appliances made therefrom, are capable of balancing seemingly contradictory properties desired by users of such appliances, and, when needed, of being adapted for limited use by users of such appliances.

FIG. 1 representatively depicts one version of a substrate 1 of the present invention. The substrate comprises an interbonded fibrous layer 3 (note: individual interbonded fiber(s), and pores between said interbonded fiber(s), are not depicted in FIG. 1) and reinforcing strands 5 attached to at least a portion of the interbonded fibrous layer. In this version of a substrate of the present invention, projections 7 emanate from the surface of the fibrous layer 3. These projections are contiguous with circular holes in and through the interbonded fibrous layer 3 (the holes in and through the interbonded fibrous layer 3 are not shown; in this representative version, the base of each projection is contiguous with such holes). Because the fibrous layer may be made up of flexible fiber, the shape of these projections may vary. For the depicted version, the projections generally have a volcano-like shape, with a larger opening at the base of the “volcano” (i.e., that portion of the projection that is contiguous with the relatively planar surface of the interbonded fibrous layer 3), and a smaller opening at the tip of the “volcano” (i.e., that portion of the projection extending away from the relatively planar surface of the interbonded fibrous layer 3). I.e., the projection is perforated at its tip. In some versions of the invention, the projection need not be perforated, but comprises more attenuated fiber such that pores between the more attenuated fiber in the projection are larger than pores between fiber in the interbonded fibrous layer not in the projection. In either case, liquid is able to move through the circular opening and any projection contiguous with said opening more readily than through the pore structure defined by the interbonded fiber not in or associated with the projection.

FIG. 1A depicts a magnified photomicrograph of a representative substrate of the present invention. The substrate comprises an interbonded fibrous layer 3 defining shaped discontinuities 8 through the thickness of the interbonded fibrous layer. The shaped discontinuities 8 are contiguous with projections 7 emanating from the surface of the interbonded fibrous layer. The projections in this image are in a somewhat flattened state, and are perforated—i.e., open—at their ends. Reinforcing strands 5 were stained so that they would contrast with the interbonded fibrous layer. FIG. 1B depicts another image of a representative substrate of the present invention. The numbers depicted in FIG. 1B signify the same elements as signified in FIGS. 1 and 1A.

As will be discussed in more detail below, the surface of the interbonded fiber layer between these projections may be textured or otherwise have three-dimensional contours imparted by the surface of the support used to form the interbonded fibrous layer, reinforcing strands, and shaped discontinuities in said interbonded fibrous layer. This is because the interbonded fibrous layer is formed on a belt (modified conveyor belts were in fact used during development of the invention) that may be selected to have a textured surface, and which includes openings (e.g., openings that are die cut, drilled, machined, molded into the belt itself, etc.). When the interbonded fibrous layer is formed on the belt, positive or negative pressure is used to force or draw the fibers near or over the openings in the belt into the openings themselves. In one version of the invention, the interbonded fibrous layer is formed by directing molten polymer to a series of spaced-apart capillaries arranged in a direction transverse to the direction of travel of the moving support on which the interbonded fibrous layer is formed. As molten polymer fiber exits these capillaries, air intermingles with the fibers and directs them to the moving support. By manipulating the positive or negative pressure used to push or draw fibers into individual openings in the belt; the temperature at which the fibrous material is formed, and therefore their susceptibility to attenuation; the polymeric raw material or ingredients used to make the molten polymer; the temperature of the air; and other such variables, the extent and degree of perforations caused by drawing and/or pushing fiber into openings in the belt may be controlled. I.e., projections analogous to those formed in FIG. 1 may be formed—volcano-like structures having an open or perforated top, and an opening at the base of the “volcano” that is contiguous with an opening in and through the interbonded fibrous layer; or projections may be formed that are not open or perforated at their top (but which have larger and more open pores due to fiber becoming more attenuated as it is pushed or pulled into an opening on the belt, thereby forming the projection), but which have an opening at the base of the “volcano” that is contiguous with an opening in and through the interbonded fibrous layer). Furthermore, fiber not drawn into the openings—i.e., that fiber contacting and carried by the surface of the belt—takes on a topography corresponding to the belt texture (if any). Thus the 3-dimensional cross section of the interbonded fibrous layer, and its surface topography on both faces, reflects: the nature of any texture on the surface of the belt; the shape, size, and placement of openings in the belt; and the choice of various process parameters, such as those mentioned above.

FIG. 2 representatively depicts one version of a personal-care appliance 10 of the present invention, in this case an appliance comprising a substrate similar to that depicted in FIG. 1. The depicted personal-care appliance is typically known as a “pouf,” “puff,” or other such similar terms. The appliance comprises an interbonded fibrous layer 12 having projections 14 emanating from the surface of the interbonded fibrous layer. These projections are contiguous with shaped discontinuities in the interbonded fibrous layer (i.e., openings in and through the interbonded fibrous layer), and these projections are perforated (i.e., open at their ends/tips). The appliance also comprises reinforcing strands 16 attached to at least a portion of the interbonded fibrous layer 12. The version of a personal-care appliance of the present invention depicted in FIG. 2 also includes a cord 18. FIG. 2A depicts an image of a representative version of a personal-care appliance of the present invention (in this case a pouf).

We have found that a pouf comprising the aforementioned substrate (i.e., an interbonded fibrous layer comprising shaped discontinuities) provides a network of pores capable of holding liquid. But this same network of pores may not provide the open area generally needed to facilitate bubble formation when a surfactant or other such soap formulation is present. Thus discontinuities may be introduced to the interbonded fibrous substrate during its formation. These discontinuities provide the open area that helps generate bubbles and foam when the personal-care appliance is squeezed or otherwise used. In effect, the resulting substrate has at least a bi-modal distribution of opening sizes: smaller pores that help hold or contain liquid, with most of these pores defined by spaces between interbonded fibers; and shaped openings that are significantly larger than said pores—e.g., with the mean diameter of pores (determined by evaluating the equivalent circular diameter of said pores, a method described in the Examples section below) typically being at least 10 to at least 100 times smaller than the size of shaped discontinuities in the interbonded fibrous layer (i.e., diameter of circular openings, when the shaped discontinuities are circular openings; or equivalent circular diameter or other analogous measure for irregular openings or openings having shapes different than a circle).

In some versions of the invention, the fiber over and near the openings in the belt is not forced or drawn into the openings such that the fiber forms a perforation in the opening. Instead fiber drawn in to the opening is attenuated such that the pores between fiber in the support openings are larger than the pores between fiber that is not within the support openings.

Because the interbonded fibrous layer comprising such shaped discontinuities may lack wet resilience (i.e., the layer may collapse and not readily spring back during use), and/or because the fibrous layer may be relatively flexible and soft, we have found that combining the interbonded fibrous layer with reinforcing strands helps to: (1) improve the wet resilience of the substrate and a personal-care appliance made therefrom; and/or (2) provide some stiffness or a somewhat unyielding component that gives some exfoliating character to the personal-care appliance.

As stated above, during development of the present invention we found that rubber belts normally used as conveyor belts could be used to form the interbonded-fibrous layer comprising shaped discontinuities. Unlike conventional forming wires, such belts are readily processed to form openings in the belt. When an interbonded fibrous layer is being formed on this support (i.e., a conveyor belt comprising openings), the fibrous layer over the openings is pulled through (in this case by a vacuum), or partially through, the openings to provide the shaped discontinuities in the fibrous layer. I.e., the shaped discontinuities in the interbonded fibrous layer corresponded to, and were formed by, the openings in the supporting belt. Conveyor belts and their analogues are also available with various textured surfaces. Thus use of such supports not only provides the ability to readily create openings of various sizes, shapes, and placement (including, for example, the cutting of recognizable shapes such as flowers, animals, a company's logo or trademark, or other such symbol or image) such that the corresponding shaped discontinuity in the interbonded fibrous layer takes on such recognizable shape in the belt.

In some versions of the present invention, a cleaning composition, moisturizing, composition, or other such formulation is injected in, or coated, sprayed, or printed on, the substrate and/or personal-care appliance. In some versions of the invention, the amount of composition associated with the substrate or personal-care appliance is such that the composition is dissipated after, for example, 1-5 uses, 1-10 uses, or some other selected value. In this way, the substrate or personal-care appliance may be adapted for limited use by the user of the substrate or appliance.

These and other versions, embodiments, and examples of the invention are discussed elsewhere in this application.

### DRAWINGS

FIG. 1 depicts a representative version of one substrate of the present invention. FIG. 1A depicts a magnified image of a representative version of one substrate of the present invention. FIG. 1B depicts an image of a representative version of one substrate of the present invention.

FIG. 2 depicts a representative version of a personal-care appliance comprising a substrate of the present invention. FIG. 2A depicts an image of a representative version of one personal-care appliance of the present invention.

FIG. 3 depicts a representative version of a process for making a substrate of the present invention.

FIGS. 4 through 7 illustrate in greater detail representative versions of forming surfaces having different textures and/or topographies. FIGS. 4A, 5A, 6A, and 7A show cross-sections taken along lines 4A-4A, 5A-5A, 6A-6A, and 7A-7A in the respective figures.

FIGS. 8A and 8B representatively illustrate a testing cell for evaluating a functional characteristic of a personal-care appliance of the present invention.

FIG. 9 representatively illustrates a test apparatus for evaluating a functional characteristic of a personal-care appliance of the present invention.

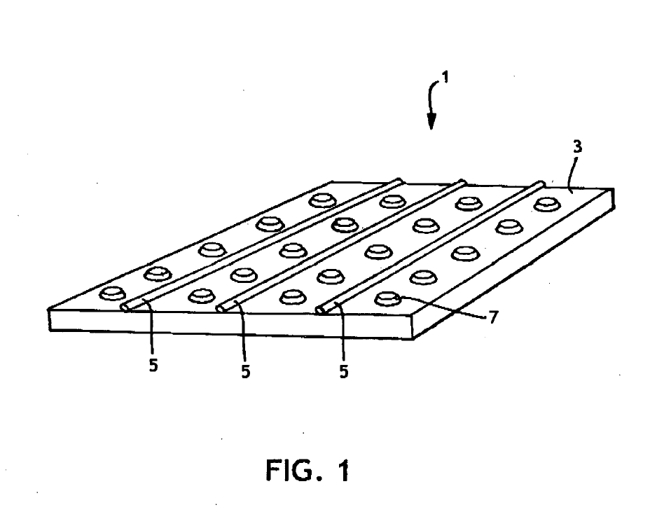

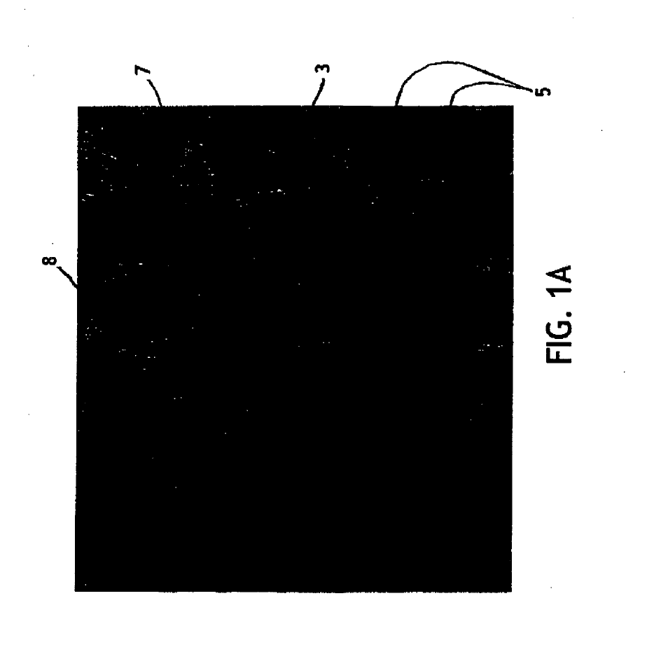

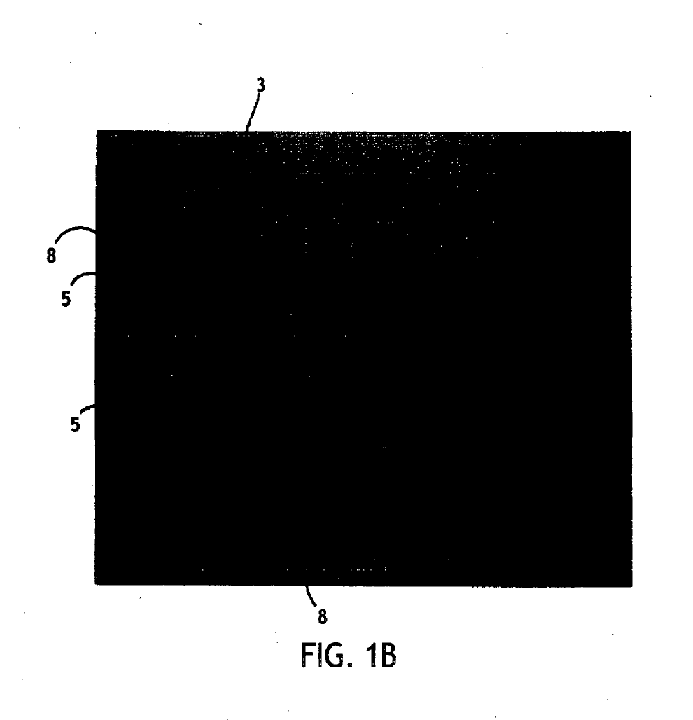

### DEFINITIONS

Within the context of this specification, each term or phrase below includes the following meaning or meanings:

“Attach” and its derivatives refer to the joining, adhering, connecting, bonding, sewing together, depositing on, associating with, or the like, of two elements. Two elements will be considered to be attached together when they are integral with one another or attached directly to one another or indirectly to one another, such as when each is directly attached to intermediate elements. “Attach” and its derivatives include permanent, releasable, or refastenable attachment. In addition, the attachment can be completed either during the manufacturing process or by the end user.

“Autogenous bonding” and its derivatives refer to bonding provided by fusion and/or self-adhesion of fibers and/or filaments without an applied external adhesive or bonding agent. Autogenous bonding may be provided by contact between fibers and/or filaments while at least a portion of the fibers and/or filaments are semi-molten or tacky. Autogenous bonding may also be provided by blending a tackifying resin with the thermoplastic polymers used to form the fibers and/or filaments. Fibers and/or filaments formed from such a blend can be adapted to self-bond with or without the application of pressure and/or heat. Solvents may also be used to cause fusion of fibers and filaments which remains after the solvent is removed.

“Bond,” “interbond,” and their derivatives refer to the joining, adhering, connecting, attaching, sewing together, or the like, of two elements. Two elements will be considered to be bonded or interbonded together when they are bonded directly to one another or indirectly to one another, such as when each is directly bonded to intermediate elements. “Bond” and its derivatives include permanent, releasable, or refastenable bonding. “Autogenous bonding,” as described above, is a type of “bonding.”

“Coform” refers to a blend of meltblown fibers and absorbent fibers such as cellulosic fibers that can be formed by air forming a meltblown polymer material while simultaneously blowing air-suspended fibers into the stream of meltblown fibers. The coform material may also include other materials, such as superabsorbent materials. The meltblown fibers and absorbent fibers are collected on a forming surface, such as provided by a belt. The forming surface may include a gas-pervious material that has been placed onto the forming surface.

“Cleaning composition”, “cleaning formulation,” or their derivatives refer to personal care or cleaning formulations or compositions, shampoos, lotions, body washes, hand sanitizers, bar soaps, etc., whether in the form of a solid, liquid, gel, paste, foam, or the like. “Cleaning compositions” also encompass moisturizing formulations.

“Connect” and its derivatives refer to the joining, adhering, bonding, attaching, sewing together, or the like, of two elements. Two elements will be considered to be connected together when they are connected directly to one another or indirectly to one another, such as when each is directly connected to intermediate elements. “Connect” and its derivatives include permanent, releasable, or refastenable connection. In addition, the connecting can be completed either during the manufacturing process or by the end user.

“Disposable” refers to articles which are designed to be discarded after a limited use rather than being laundered or otherwise restored for reuse.

The terms “disposed on,” “disposed along,” “disposed with,” or “disposed toward” and variations thereof are intended to mean that one element can be integral with another element, or that one element can be a separate structure bonded to or placed with or placed near another element.

“Fiber” refers to a continuous or discontinuous member having a high ratio of length to diameter or width. Thus, a fiber may be a filament, a thread, a strand, a yarn, or any other member or combination of these members.

“Hydrophilic” describes fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90 degrees are designated “wettable” or hydrophilic, and fibers having contact angles greater than 90 degrees are designated “nonwettable” or hydrophobic.

“Layer” when used in the singular can have the dual meaning of a single element or a plurality of elements.

“Liquid impermeable,” when used in describing a layer or multi-layer laminate means that liquid will not pass through the layer or laminate, under ordinary use conditions, in a direction generally perpendicular to the plane of the layer or laminate at the point of liquid contact.

“Liquid permeable” refers to any material that is not liquid impermeable.

“Meltblown” refers to fibers formed by extruding a molten thermoplastic material through a plurality of fine, usually circular, die capillaries as molten threads or filaments into converging high velocity gas (e.g., air) streams, generally heated, which attenuate the filaments of molten thermoplastic material to reduce their diameters. Thereafter, the meltblown fibers are carried by the high velocity gas stream and are deposited on a collecting surface or support to form a web of randomly dispersed meltblown fibers. Such a process is disclosed, for example, in U.S. Pat. No. 3,849,241 to Butin et al. Meltblowing processes can be used to make fibers of various dimensions, including macrofibers (with average diameters from about 40 to about 100 microns), textile-type fibers (with average diameters between about 10 and 40 microns), and microfibers (with average diameters less than about 10 microns). Meltblowing processes are particularly suited to making microfibers, including ultra-fine microfibers (with an average diameter of about 3 microns or less). A description of an exemplary process of making ultra-fine microfibers may be found in, for example, U.S. Pat. No. 5,213,881 to Timmons, et al. Meltblown fibers may be continuous or discontinuous and are generally self bonding when deposited onto a collecting surface.

“Member” when used in the singular can have the dual meaning of a single element or a plurality of elements.

“Nonwoven” and “nonwoven web” refer to materials and webs of material that are formed without the aid of a textile weaving or knitting process. For example, nonwoven materials, fabrics or webs have been formed from many processes such as, for example, meltblowing processes, spunbonding processes, air laying processes, coform processes, and bonded carded web processes.

Descrption

Representative Process for Making Substrate of Present Invention

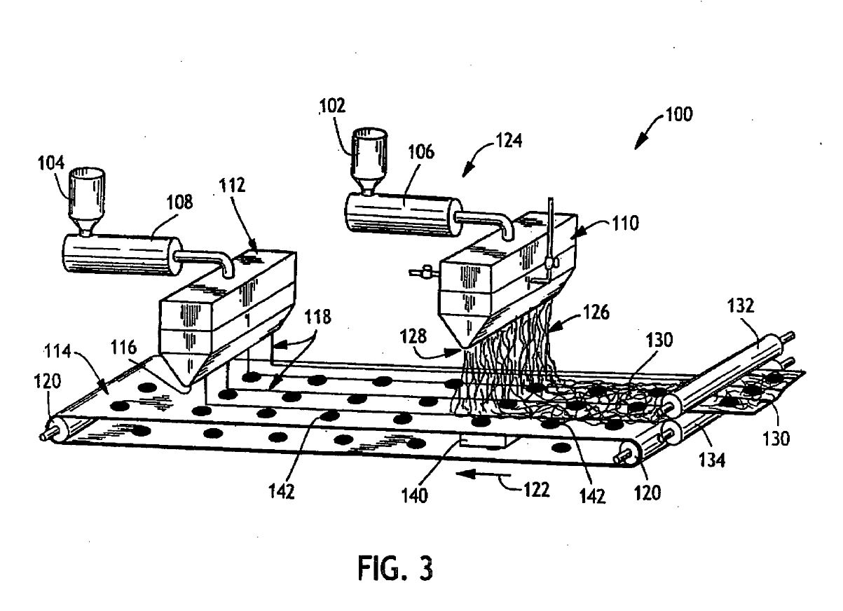

FIG. 3 is a representative schematic view of a process for forming a substrate of the present invention. The process is generally represented by reference numeral 100. In forming the interbonded fibrous layer and the reinforcing strands which are used in the substrate, pellets or chips, etc. (not shown) of an extrudable polymer are introduced into pellet hoppers 102 and 104 of extruders 106 and 108.

Each extruder has an extrusion screw (not shown) which is driven by a conventional drive motor (not shown). As the polymer advances through the extruder, due to rotation of the extrusion screw by the drive motor, it is progressively heated to a molten state. Heating the polymer to the molten state may be accomplished in a plurality of discrete steps with its temperature being gradually elevated as it advances through discrete heating zones of the extruder 106 toward a meltblowing die 110 and extruder 108 toward a continuous strand forming means 112 (i.e., a reinforcing strand forming means). The meltblowing die 110 and the continuous strand forming means 112 may be yet another heating zone where the temperature of the thermoplastic resin is maintained at an elevated level for extrusion. Heating of the various zones of the extruders 106 and 108 and the meltblowing die 110 and the continuous strand forming means 112 may be achieved by any of a variety of conventional heating arrangements (not shown).

The reinforcing strand component of the substrate may be formed utilizing a variety of extrusion techniques. For example, the reinforcing strands may be formed utilizing one or more conventional meltblowing die arrangements which have been modified to remove the heated gas stream (i.e., the primary air stream) which would otherwise flow generally in the same direction as that of the extruded strands to attenuate the extruded strands. This modified meltblowing die arrangement 112 usually extends across a collecting surface or support 114 in a direction which is substantially transverse to the direction of movement of the collecting surface or support 114. The modified die arrangement 112 includes a linear array 116 of small diameter capillaries aligned along the transverse extent of the die with the transverse extent of the die being approximately as long as the desired width of the parallel rows (or other alignment) of reinforcing strands which are to be produced. That is, the transverse dimension of the die is the dimension which is defined by the linear array of die capillaries. The diameter of the capillaries may be on the order of from about 0.01 inches to about 0.02 inches, or, for example, from about 0.0145 to about 0.018 inches. But larger diameter capillaries may be used to enhance the exfoliating characteristics of the interbonded fibrous layer, to help reinforce the interbonded fibrous layer, or both. Thus the reinforcing strands may be significantly larger (e.g., the reinforcing strands may be extruded through capillaries having a diameter of between about 0.020 inches and about 0.050 inches, or even larger). In Example 1 below, the reinforcing strands are extruded through capillaries having a diameter of 0.050 inches. From about 1 to about 50 such capillaries will be provided per linear inch of die face. Typically, the length of the capillaries will be from about 0.05 inches to about 0.20 inches, for example, about 0.113 inches to about 0.14 inches long. A meltblowing die can extend from about 10 inches to about 60 or more inches in length in the transverse direction.

Since the heated gas stream (i.e., the primary air stream) which flows past the die tip is greatly reduced or absent, it may be desirable to insulate the die tip or provide heating elements to ensure that the extruded polymer remains molten and flowable while in the die tip. Polymer is extruded from the array 116 of capillaries in the modified die 112 to create extruded reinforcing strands 118.

The extruded reinforcing strands 118 have an initial velocity as they leave the array 116 of capillaries in the modified die 112. These strands 118 are deposited upon a surface 114 which should be moving at least at the same velocity as the initial velocity of the strands 118. This surface or support 114 is an endless belt conventionally driven by rollers 120. In the depicted representative embodiment, the strands 118 are deposited in substantially parallel alignment on the surface of the endless belt 114 which is rotating as indicated by the arrow 122 in FIG. 3. Vacuum boxes (not shown) may be used to assist in retention of the matrix on the surface of the belt 114. The tip of the die 112 should be as close as practical to the surface of the belt 114 upon which the reinforcing strands 118 are collected. For example, this forming distance may be from about 1 inch to about 10 inches. Desirably, this distance is from about 1 inch to about 8 inches.

It may be desirable to have the surface 114 moving at a speed that is much greater than the initial velocity of the reinforcing strands 118 in order to enhance the alignment of the strands 118 into substantially parallel rows and/or elongate the filaments 118 so they achieve a desired diameter. For example, alignment of the strands 118 may be enhanced by having the surface 114 move at a velocity from about 2 to about 10 times greater than the initial velocity of the strands 118. Even greater speed differentials may be used if desired. While different factors will affect the particular choice of velocity for the surface 114, it will typically be from about four to about eight times faster than the initial velocity of the reinforcing strands 118.

Desirably, the reinforcing strands are formed at a density per inch of width of material which corresponds generally to the density of capillaries on the die face. For example, the strand density per inch of width of material may range from about 1 to about 120 such filaments per inch width of material. Typically, lower densities of filaments (e.g., 1-35 filaments per inch of width) may be achieved with only one strand forming die. Higher densities (e.g., 35-120 strands per inch of width) may be achieved with multiple banks of strand-forming equipment.

While the reinforcing strands are depicted as being essentially parallel in the embodiment depicted in FIG. 3, they need not be so. For example, the belt, the bank of dies, or some combination thereof could oscillate or move such that the strands trace out, for example, a sinusoidal wave or other pattern. Furthermore, the strands may be discontinuous, rather than continuous. Also, as discussed below, the reinforcing fibers could be introduced in a manner analogous to the manner in which the interbonded fibrous layer is formed. In other words, reinforcing strands may be introduced as a meltblown material, but using different polymeric raw material(s), die-capillary diameters, air temperatures (i.e., as is discussed below, the temperature of the air used to intermingle and otherwise conduct the strands/fibers exiting the die capillaries toward, and on to, the support), temperature of the molten polymer exiting the die capillaries, etc.

In the representative version of FIG. 3, the interbonded fibrous layer is meltblown fiber. Here the meltblown fiber component of the substrate is formed utilizing a conventional meltblowing process represented by reference numeral 124. Meltblowing processes generally involve extruding a thermoplastic polymer resin through a plurality of small diameter capillaries of a meltblowing die as molten threads into a heated gas stream (the primary air stream) which is flowing generally in the same direction as that of the extruded threads so that the extruded threads are attenuated, i.e., drawn or extended, to reduce their diameter. Such meltblowing techniques and apparatus are discussed fully in U.S. Pat. No. 4,663,220, which is hereby incorporated by reference in its entirety in a manner consistent herewith.

In the meltblown die arrangement 110, the position of air plates which, in conjunction with a die portion define chambers and gaps, may be adjusted relative to the die portion to increase or decrease the width of the attenuating gas passageways so that the volume of attenuating gas passing through the air passageways during a given time period can be varied without varying the velocity of the attenuating gas. Generally speaking, lower attenuating gas velocities and wider air passageway gaps are generally preferred if substantially continuous meltblown fibers or microfibers are to be produced.

The two streams of attenuating gas converge to form a stream of gas which entrains and attenuates the molten threads, as they exit the orifices, into fibers, depending upon the degree of attenuation, microfibers, of a small diameter which is usually less than the diameter of the orifices. The gas-borne fibers or microfibers 126 are blown, by the action of the attenuating gas, onto a collecting arrangement which, in the embodiment illustrated in FIG. 3, is the endless belt 114 which carries the reinforcing strand in substantially parallel alignment. The fibers or microfibers 126 are collected as a coherent matrix of fibers on the surface of the reinforcing strands 118 and endless belt 114 which is rotating as indicated by the arrow 122 in FIG. 3. If desired, the meltblown fibers or microfibers 126 may be collected on the endless belt 114 at numerous impingement angles. A vacuum box 140 is used to draw the meltblown fibers into the openings 142 in the endless belt or support 114. By adjusting process parameters (e.g., amount of vacuum; temperature at which meltblown fibers exit the orifices), the interbonded fibrous layer is drawn into the openings in the support 114 so that shaped discontinuities are formed in the interbonded fibrous layer itself. I.e., the shaped discontinuities in the interbonded fibrous layer correspond to the openings in support 114. It should be noted that this forming process does not create the amount of waste inherent in cutting holes or other openings directly in the interbonded fibrous layer (if the projections are perforated or open at their tips). In the present invention, the meltblown fibers proximate to (i.e., over or near) openings 142 are further attenuated by the action of the vacuum drawing the fiber into the openings. A portion of the attenuated fiber within the openings separate, thereby forming perforations or openings at the tip of any projection emanating from the surface of the interbonded fibrous layer (and contiguous with the shaped opening in the interbonded fibrous layer itself).

It should be noted that the depicted openings 142 in the support 114 in FIG. 3 are representative. The shape, size, number, and placement of such openings can be varied. For example, the openings in the belt may be rectangles, squares, triangles, ovals, stars, crosses, pentagons, hexagons, octagons, other such geometric shapes, and various combinations thereof. Furthermore, the openings, die cut or otherwise, may be more complex, and in fact may depict various recognizable living or non-living objects. For example, an opening defining the shape of a teddy bear might be used. Or an opening defining the shape of a tulip, air plane, rocket, or any number of other such objects might be used. Or, as mentioned above, a company's logo, tradename, or trademark might be introduced to the support 114, with the corresponding image introduced to the interbonded fibrous layer.

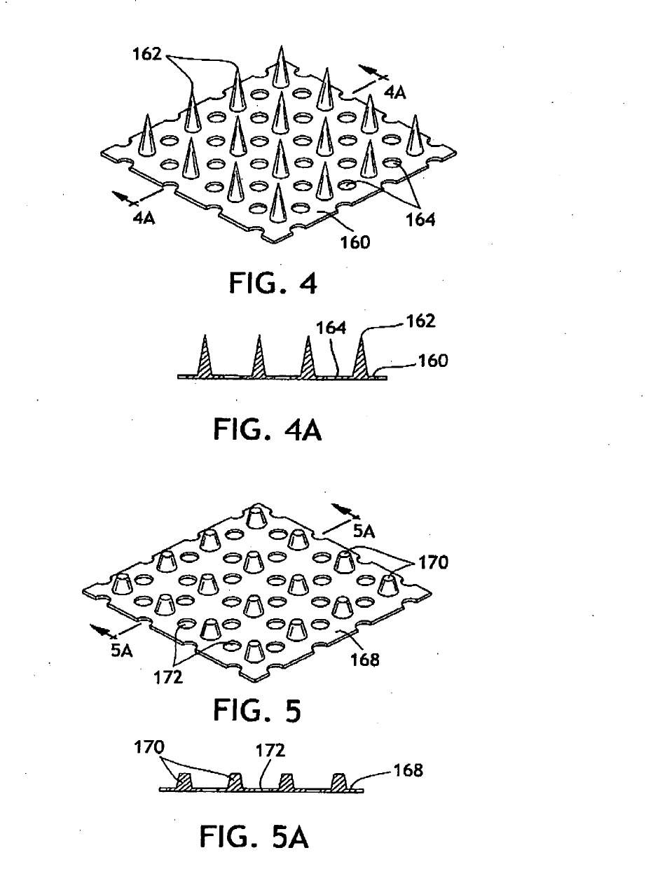

It should also be noted that the surface of the belt itself may be textured. Examples of various textured surfaces include a pebbled surface; a surface having the appearance of a molded screen—with individual strands interleaved with one another; a surface having the appearance of a lattice with diamond-shaped openings; etc. Furthermore, the textured surface may have a complex surface topography, with multiple tiers. The thickness of the belt may be varied to accommodate the selected texture on the surface of the belt and the selected openings in the belt. A few representative versions of such textures are depicted in FIGS. 4, 4A, 5, 5A, 6, 6A, 7, and 7A.

FIG. 4 illustrates in greater detail and in perspective view one forming surface which can be used as belt 114 in FIG. 3. As shown, the surface in this case is a flat belt 160 having cone-shaped pins 162 which are disposed outwardly from the surface. In this embodiment belt 160 also contains openings 164. FIG. 4A shows the forming surface of FIG. 4 in cross-section taken along lines 4A-4A. The forming surface in FIG. 4 could be used without the cone-shaped pins 162, and could further include different textures or surface topographies between the openings 164. As noted above, the openings may be of a variety of shapes other than circles, and the placement of these openings can be varied as desired. Although in the representative embodiment depicted in FIGS. 4 and 4A the openings have a uniform diameter through the thickness of the belt, the openings in the belt may be fashioned to have a changing diameter through the thickness of the belt.

FIG. 5 is a view of an alternative forming surface 168 which, in this case, has pins 170 in the shape of truncated cones extending outwardly and openings 172. FIG. 5A is a cross-section of the surface of FIG. 5 taken along lines 5A-5A. The forming surface in FIG. 5 could be used without the cone-shaped pins 170, and could further include different textures or surface topographies between the openings 172. Also, if used, the pins could be further truncated to varying degrees short of total elimination of the pins. As noted above, the openings may be of a variety of shapes other than circles, and the placement of these openings can be varied as desired. Although in the representative embodiment depicted in FIGS. 5 and 5A the openings have a uniform diameter through the thickness of the belt, the openings in the belt may be fashioned to have a changing diameter through the thickness of the belt.

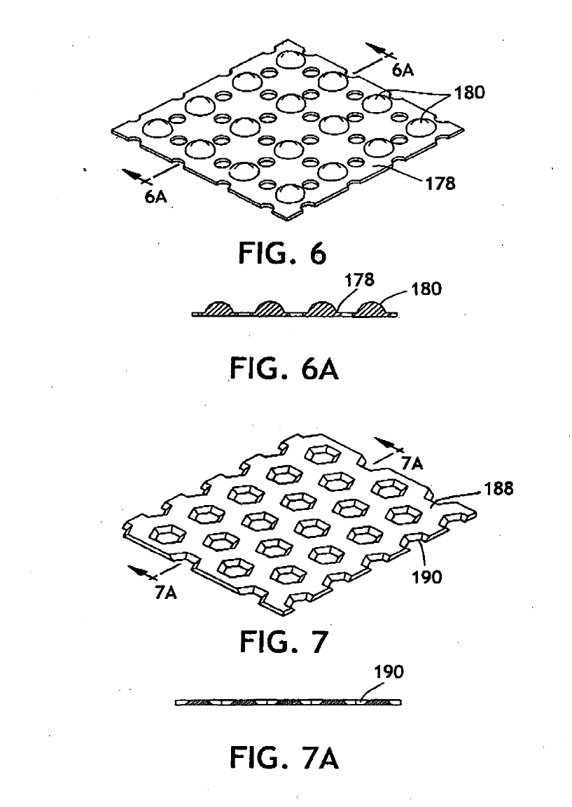

FIGS. 6 and 6A are views like FIGS. 4 and 4A illustrating yet other forming surfaces 178 having domes 180 at the surface of the belt.

FIG. 7 illustrates an alternative belt configuration 188, in this case comprising hexagonal openings 190, useful in making an interbonded fibrous layer of the present invention, and FIG. 7A shows the belt of FIG. 7 in cross-section taken along lines 7A-7A. As noted earlier, openings need not have a uniform cross-section through the thickness of the belt. FIG. 7A shows that the interior surfaces of the hexagon slope inward to the center of the hexagon itself. Openings also may have multiple tiers through the thickness of the belt. I.e., the inner diameter (or other distance depending on the shape of the opening) may change in a step-wise fashion through the thickness of the belt (rather than in a monotonically increasing or decreasing fashion).

The present invention encompasses many other such textured surfaces or three- dimensional topographies on belts or supports, said textured surface or topography imparting a corresponding three-dimensional topography to the interbonded fibrous layer. It should be noted that “three-dimensional topography” here signifies a topography readily discernible by the human eye (e.g., changes in elevation of about 0.1 millimeter or more—suitably of about 0.5 millimeter or more—from the base of a “valley” to the top of a neighboring “ridge” in the surface of the interbonded fibrous layer; a “valley” signifies a low point or depression in the first interbonded fibrous layer; a “ridge” signifies a high point or elevation in the first interbonded fibrous layer). Such topographies are contrasted with the topography associated with a flat sheet of writing paper, or a flat, unembossed sheet of toilet tissue. Such substrates, under a microscope, reveal surfaces having a microscopic three-dimensional topography. But such topographies are to be distinguished from the three-dimensional topographies discussed herein with respect to surfaces of interbonded fibrous layers.

Vacuum boxes, such as that identified in the drawing by numeral 140, may be used to assist generally in retention of the matrix on the surface of the belt 114. Typically the tip 128 of the die 110 is from about 6 inches to about 14 inches from the surface of the belt 114 upon which the fibers are collected. The entangled fibers or microfibers 124 autogenously bond to at least a portion of the reinforcing strands 118 because the fibers or microfibers 124 are still somewhat tacky or molten while they are deposited on the reinforcing strands 118, thereby forming the substrate 130.

At this point, it may be desirable to lightly calender the substrate in order to enhance the autogenous bonding. This optional calendering step may be accomplished with a pair of patterned or un-patterned pinch rollers 132 and 134 under sufficient pressure (and temperature, if desired) to help facilitate autogenous bonding between the reinforcing strands and the interbonded fibrous layer (here a meltblown layer).

As discussed above, the reinforcing strands and interbonded fibrous layer are deposited upon a moving surface (e.g., support 114 in the representative version of a process depicted in FIG. 3). In one embodiment of the invention, meltblown fibers are formed directly on top of the extruded reinforcing strands. This is achieved by passing the strands and the surface under equipment which produces the interbonded fibrous layer (meltblown material in the version of the process depicted in FIG. 3). Alternatively, an interbonded fibrous layer, such as a meltblown material, may be deposited on a surface and substantially parallel rows (or other alignment) of reinforcing strands may be formed directly upon the interbonded fibrous layer. Various combinations of strand-forming and fiber-forming equipment may be set up to produce different types of substrates. For example, the substrate may contain alternating layers of reinforcing strands and interbonded fibrous layers. Several dies for forming interbonded fibrous layers or creating reinforcing strands may also be arranged in series to provide superposed layers of fibers or strands.

The location of the means for forming the reinforcing strands relative to the location of the means for forming the interbonded fibrous layer may be selected (taking into consideration the range of velocities at which support 114 moves) to obtain desired time intervals between the time at which the reinforcing strands are extruded and the time at which the interbonded fibrous layer contacts the reinforcing strands (or vice versa, if the interbonded fibrous layer is formed first, and the reinforcing strands are extruded onto the interbonded fibrous layer). Typically the time interval will allow for the reinforcing strands, the interbonded fibrous layer, or both, to be somewhat tacky and to be capable of autogenous bonding. Note, however, that an adhesive could be applied to the reinforcing strands, the interbonded fibrous layer, or both to promote bonding.

As noted above, the invention contemplates multiple banks of dies for forming the interbonded fibrous layer, the reinforcing strands, or both. Furthermore, the individual capillaries within a linear array of said capillaries; between multiple banks of linear arrays of capillaries; or both, may be of different sizes. Also, the operating parameters for a given linear array of capillaries (e.g., temperature at which the molten polymer exits the capillaries; velocity and/or temperature of any air flow used to carry and/or attenuate the exiting fiber or strand; the identity of the polymeric raw material(s); etc.) may be different across said linear array; between multiple banks of linear arrays of capillaries; or both.

Representative Materials with which the Reinforcing Strand and/or Interbonded Fibrous Layer may be Made

The interbonded fibrous layer and reinforcing strands may be made from any material which may be manufactured into such fibrous layer and strands. For those personal-care appliances requiring or benefiting from elastomeric characteristics, the substrate may be made using suitable elastomeric fiber-forming resins or blends containing the same for the interbonded fibrous layer; and any suitable elastomeric strand-forming resins or blends containing the same may be utilized for the reinforcing strands. The interbonded fibers and filaments may be formed from the same or different elastomeric resin.

For example, the interbonded fibrous layer and/or the reinforcing strands may be made from block copolymers having the general formula A-B-A′ where A and A′ are each a thermoplastic polymer endblock which contains a styrenic moiety such as a poly (vinyl arene) and where B is an elastomeric polymer midblock such as a conjugated diene or a lower alkene polymer. The block copolymers may be, for example, (polystyrene/poly(ethylene-butylene)/polystyrene) block copolymers available from the Shell Chemical Company under the trademark KRATON. G. One such block copolymer may be, for example, KRATON G-1657.

Other exemplary materials which may be used include polyurethane materials such as, for example, those available under the trademark ESTANE from B. F. Goodrich & Co., polyamide materials such as, for example, those available under the trademark PEBAX from the Rilsan Company, and polyester materials such as, for example, those available under the trade designation Hytrel from E. I. DuPont De Nemours & Company. Formation of meltblown fibers from polyester materials is disclosed in, for example, U.S. Pat. No. 4,741,949 to Morman et al., which is hereby incorporated by reference in its entirety in a manner consistent herewith. Useful polymers also include, for example, copolymers of ethylene and at least one vinyl monomer such as, for example, vinyl acetates, unsaturated aliphatic monocarboxylic acids, and esters of such monocarboxylic acids. The copolymers and formation of meltblown fibers from those copolymers are disclosed in, for example, U.S. Pat. No. 4,803,117.

Processing aids may be added to the polymer. For example, a polyolefin may be blended with the polymer (e.g., the A-B-A elastomeric block copolymer) to improve the processability of the composition. The polyolefin must be one which, when so blended and subjected to an appropriate combination of elevated pressure and elevated temperature conditions, extrudable, in blended form, with the polymer. Useful blending polyolefin materials include, for example, polyethylene, polypropylene and polybutene, including ethylene copolymers, propylene copolymers and butene copolymers. A particularly useful polyethylene may be obtained from the U.S.I. Chemical Company under the trade designation Petrothene NA 601 (also referred to herein as PE NA 601 or polyethylene NA 601). Two or more of the polyolefins may be utilized. Extrudable blends of polymers and polyolefins are disclosed in, for example, previously referenced U.S. Pat. No. 4,663,220.

Desirably, the interbonded fibrous layer and/or the reinforcing strands should have some tackiness or adhesiveness to enhance autogenous bonding. For example, the polymer itself may be tacky when formed into fibers and/or strands or, alternatively, a compatible tackifying resin may be added to the extrudable compositions described above to provide tackified fibers and/or strands that autogenously bond. In regard to the tackifying resins and tackified extrudable compositions, note the resins and compositions as disclosed in U.S. Pat. No. 4,787,699, hereby incorporated by reference in its entirety in a manner consistent herewith.

Any tackifier resin can be used which is compatible with the polymer and can withstand the processing (e.g., extrusion) temperatures. If the polymer (e.g., A-B-A elastomeric block copolymer) is blended with processing aids such as, for example, polyolefins or extending oils, the tackifier resin should also be compatible with those processing aids. Generally, hydrogenated hydrocarbon resins are preferred tackifying resins, because of their better temperature stability. REGALREZ and ARKON series tackifiers are examples of hydrogenated hydrocarbon resins. ZONATAK 501 lite is an example of a terpene hydrocarbon. REGALREZ hydrocarbon resins are available from Hercules Incorporated. ARKON series resins are available from Arakawa Chemical (U.S.A.) Incorporated. Of course, the present invention is not limited to use of such three tackifying resins, and other tackifying resins which are compatible with the other components of the composition and can withstand the processing temperatures, can also be used.

Typically, the blend used to form the reinforcing strands and/or interbonded fibers for the interbonded fibrous layer include, for example, from about 40 to about 80 percent by weight polymer, from about 5 to about 40 percent polyolefin and from about 5 to about 40 percent resin tackifier. For example, a particularly useful composition included, by weight, about 61 to about 65 percent KRATON G-1657, about 17 to about 23 percent polyethylene NA 601, and about 15 to about 20 percent REGALREZ 1126.

The interbonded fibrous layer component of a substrate of the present invention may be a mixture of elastic and nonelastic fibers or particulates. For an example of such a mixture, reference is made to U.S. Pat. No. 4,209,563, which is hereby incorporated by reference in its entirety in a manner consistent herewith, in which elastomeric and non-elastomeric fibers are commingled to form a single coherent web of randomly dispersed fibers. Another example of such an composite web would be one made by a technique such as disclosed in previously referenced U.S. Pat. No. 4,741,949. That patent discloses an elastic nonwoven material which includes a mixture of meltblown thermoplastic fibers and other materials. The fibers and other materials are combined in the gas stream in which the meltblown fibers are borne so that an intimate entangled commingling of meltblown fibers and other materials, e.g., wood pulp, staple fibers or particulates such as, for example, activated charcoal, clays, starches, or hydrocolloid (hydrogel) particulates commonly referred to as super-absorbents occurs prior to collection of the fibers upon a collecting device to form a coherent web of randomly dispersed fibers.

To give the substrate, and any personal-care appliances made therefrom, increased wet resilience, strength, and/or exfoliating character, the reinforcing strands may be made from a polyolefin such as polypropylene. Particularly suitable polymers for forming the reinforcing fiber include polypropylene and copolymers of polypropylene and ethylene. Other polymers useful in the manufacture of reinforcing strand (and/or the interbonded fibrous layer) may further include thermoplastic polymers like polyolefins, polyesters and polyamides. Elastic polymers may also be used and include block copolymers such as polyurethanes, copolyether esters, polyamide polyether block copolymers, ethylene vinyl acetates (EVA), block copolymers having the general formula A-B-A′ or A-B like copoly(styrene/ethylene-butylene), styrene-poly(ethylene-propylene)-styrene, styrene-poly(ethylene-butylene)-styrene, (polystyrene/poly(ethylene-butylene)/polystyrene, poly(styrene/ethylene-butylene/styrene) and the like.

Polyolefins using single site catalysts, sometimes referred to as metallocene catalysts, may also be used to make the interbonded fibrous layer and/or the reinforcing strands. Many polyolefins are available for fiber production, for example polyethylenes such as Dow Chemical's ASPUN7 6811A linear low density polyethylene, 2553 LLDPE and 25355 and 12350 high density polyethylene are such suitable polymers. The polyethylenes have melt flow rates, respectively, of about 26, 40, 25 and 12. Fiber forming polypropylenes include Exxon Chemical Company's 3155 polypropylene and Montell Chemical Co.'s PF-304 and/or PF-015. Many other polyolefins are commercially available.

Biodegradable polymers are also available for interbonded fiber and reinforcing strand production and suitable polymers include polylactic acid (PLA) and a blend of BIONOLLE, adipic acid and UNITHOX (BAU). PLA is not a blend but a pure polymer like polypropylene. BAU represents a blend of BIONOLLE, adipic acid, and UNITHOX at different percentages. Typically, the blend for staple fiber is 44.1 percent BIONOLLE 1020, 44.1 percent BIONOLLE 3020, 9.8 percent adipic acid and 2 percent UNITHOX 480, though spunbond BAU fibers typically use about 15 percent adipic acid. BIONOLLE 1020 is polybutylene succinate, BIONOLLE 3020 is polybutylene succinate adipate copolymer, and UNITHOX 480 is an ethoxylated alcohol. BIONOLLE is a trademark of Showa Highpolymer Co. of Japan. UNITHOX is a trademark of Baker Petrolite which is a subsidiary of Baker Hughes International.

Polypropylene, and other such polymeric materials, generally make for a stiffer, stronger fiber, especially if, as described above, the reinforcing strands are extruded with a larger diameter compared to the diameter of the fibers in the interbonded fibrous layer. Furthermore, the polymeric materials from which the reinforcing strand is made can be selected so that the reinforcing strands soften at a temperature higher than the temperature at which the interbonded fibrous layer softens. For those embodiments where reinforcing strands are extruded over openings in support 114, selection of the material, or materials of construction, of the reinforcing strands such that the strands have a softening point higher than that of the interbonded fibrous layer can help ensure that the reinforcing strands are not pulled into the openings 140 when a vacuum 142 is applied. Alternatively, the location of the small diameter capillaries along the transverse dimension of the die may be selected such that the reinforcing strands are not extruded over openings in the support.

Representative Personal-Care Appliance Comprising a Substrate of the Present Invention

Various personal-care appliances may be prepared or converted from the substrate disclosed above. The substrate can be provided as a flat sheet, or in rolled form, as a towel-like or hand-cloth-like personal-care appliance.

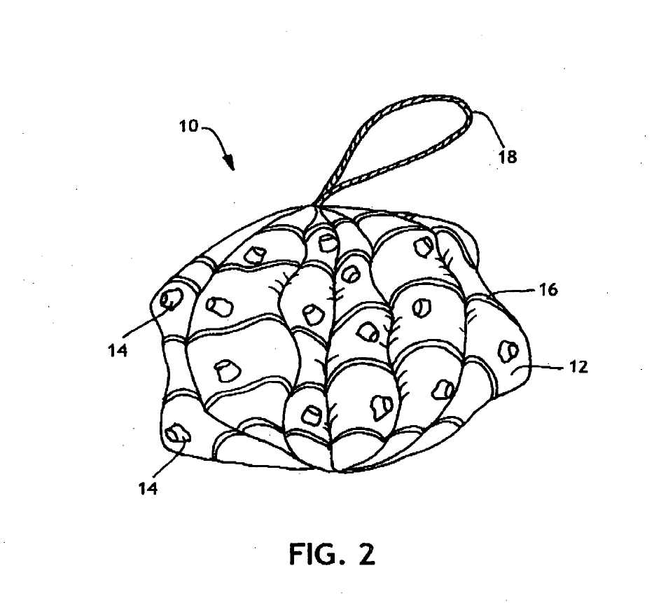

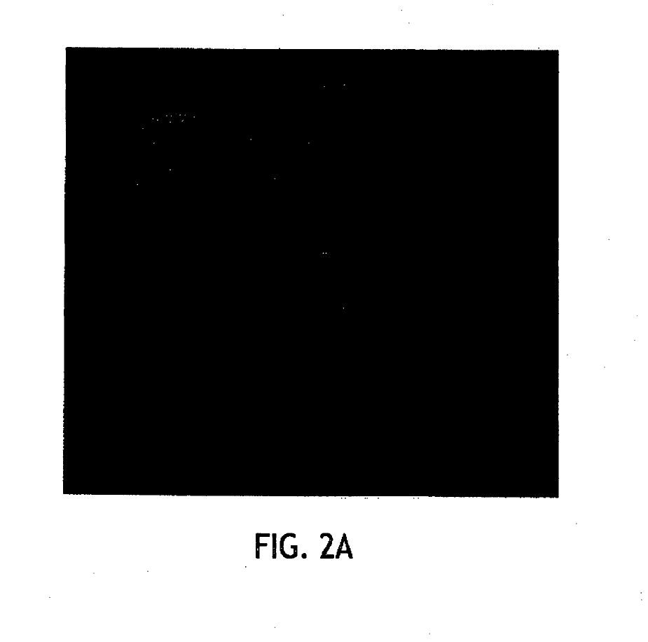

Alternatively, the substrate can be provided as a flat sheet along with a cord so that the flat sheet (or sheets) can be combined with the cord to make a pouf analogous to that depicted in FIG. 2. Various personal-care appliances of this nature, as well as methods of making such personal care appliances, are described in U.S. patent application Ser. No. 04/011,739, entitled “Disposable and Reusable Pouf Products” and listing R. Dilnik, et al., as inventors. This reference is hereby incorporated by reference in its entirety in a manner consistent herewith.

A personal-care appliance may also be converted from the substrate such that the appliance is generally spherical, cylindrical, or other such shape, and is available as such. In one version of making such an appliance (i.e., converting the substrate into an appliance), the ends of a given length of the substrate are joined, bonded, or attached to one another such that a continuous loop of the substrate is formed. The loop is then stretched so that it fits over two supports. After fusing, joining, or binding the central part of the loop (generally mid way between the two supports), the loop is removed from the support to form a generally spherical personal-care appliance. Substantially spherical objects made in this fashion are described in, for example, U.S. Pat. Nos. 2,666,249 and 3,816,888. It should be noted that a personal-care appliance of this type may be made with the reinforcing strands oriented outward such that the strands are adapted to contact the skin or other body surface of a user of the appliance. Alternatively, the personal-care appliance may be made such that the reinforcing strands are oriented inward toward the interior of the pouf (i.e., away from, and not available to contact, the skin or other body surface of a user of the appliance).

Representative Cleaning Compositions that may be Deposited on a Substrate or Personal-Care Appliance of the Present Invention

Cleaning compositions that may be deposited on or otherwise associated with substrates and/or personal-care appliances of the present invention include soaps, skin lotions, colognes, sunscreens, shampoos, gels, bodywashes, and the like. Such compositions may be in solid, liquid, gel, foam, or other forms. Such compositions may also include, or be, moisturizing agents or formulations.

Many cleaning compositions contain similar core ingredients; such as water and surfactants. They may also contain oils, detergents, emulsifiers, film formers, waxes, perfumes, preservatives, emollients, solvents, thickeners, humectants, chelating agents, stabilizers, pH adjusters, and so forth. In U.S. Pat. No. 3,658,985, for example, an anionic based composition contains a minor amount of a fatty acid alkanolamide. U.S. Pat. No. 3,769,398 discloses a betaine-based composition containing minor amounts of nonionic surfactants. U.S. Pat. No. 4,329,335 also discloses a composition containing a betaine surfactant as the major ingredient and minor amounts of a nonionic surfactant and of a fatty acid mono- or di-ethanolamide. U.S. Pat. No. 4,259,204 discloses a composition comprising 0.8 to 20% by weight of an anionic phosphoric acid ester and one additional surfactant which may be either anionic, amphoteric, or nonionic. U.S. Pat. No. 4,329,334 discloses an anionic amphoteric based composition containing a major amount of anionic surfactant and lesser amounts of a betaine and nonionic surfactants.

U.S. Pat. No. 3,935,129 discloses a liquid cleaning composition containing an alkali metal silicate, urea, glycerin, triethanolamine, an anionic detergent and a nonionic detergent. The silicate content determines the amount of anionic and/or nonionic detergent in the liquid cleaning composition. U.S. Pat. No. 4,129,515 discloses a liquid detergent comprising a mixture of substantially equal amounts of anionic and nonionic surfactants, alkanolamines and magnesium salts, and, optionally, zwitterionic surfactants as suds modifiers. U.S. Pat. No. 4,224,195 discloses an aqueous detergent composition comprising a specific group of nonionic detergents, namely, an ethylene oxide of a secondary alcohol, a specific group of anionic detergents, namely, a sulfuric ester salt of an ethylene oxide adduct of a secondary alcohol, and an amphoteric surfactant which may be a betaine, wherein either the anionic or nonionic surfactant may be the major ingredient. Detergent compositions containing all nonionic surfactants are shown in U.S. Pat. Nos. 4,154,706 and 4,329,336. U.S. Pat. No. 4,013,787 discloses a piperazine based polymer in conditioning and shampoo compositions. U.S. Pat. No. 4,450,091 discloses high viscosity compositions containing a blend of an amphoteric betaine surfactant, a polyoxybutylenepolyoxyethylene nonionic detergent, an anionic surfactant, a fatty acid alkanolamide and a polyoxyalkylene glycol fatty ester. U.S. Pat. No. 4,595,526 describes a composition comprising a nonionic surfactant, a betaine surfactant, an anionic surfactant and a C12-C14 fatty acid mono-ethanolamide foam stabilizer. The contents of the patents discussed herein are hereby incorporated by reference as if set forth in their entirety and in a manner consistent herewith.

Further information on these ingredients may be obtained, for example, by reference to: Cosmetics & Toiletries, Vol. 102, No. 3, March 1987; Balsam, M. S., et al., editors, Cosmetics Science and Technology, 2nd edition, Vol. 1, pp 27-104 and 179-222 Wiley-Interscience, New York, 1972, Vol. 104, pp 67-111, February 1989; Cosmetics & Toiletries, Vol. 103, No. 12, pp 100-129, December 1988, Nikitakis, J. M., editor, CTFA Cosmetic Ingredient Handbook, first edition, published by The Cosmetic, Toiletry and Fragrance Association, Inc., Washing-ton, D.C., 1988, Mukhtar, H, editor, Pharmacology of the Skin, CRC Press 1992; and Green, F J, The Sigma-Aldrich Handbook of Stains. Dyes and Indicators; Aldrich Chemical Company, Milwaukee Wis., 1991, the contents of which are hereby incorporated by reference as if set forth in their entirety and in a manner consistent herewith.

Exemplary materials that may be used in the practice of this invention further include but are not limited to those discussed in Cosmetic and Toiletry Formulations by Ernest W. Flick, ISBN 0-8155-1218-X, second edition, section XII (pages 707-744).

Other ingredients that may be included in a composition or formulation associated with a substrate or personal-care appliance of the present invention include emulsifiers, surfactants, viscosity modifiers, natural moisturizing factors, antimicrobial actives, pH modifiers, enzyme inhibitors/inactivators, suspending agents, pigments, dyes, colorants, buffers, perfumes, antibacterial actives, antifungal actives, pharmaceutical actives, film formers, deodorants, opacifiers, astringents, solvents, organic acids, preservatives, drugs, vitamins, aloe vera, some combination thereof, and the like.

Such compositions and formulations may be applied to, on, or otherwise associated with the substrate and/or personal-care appliance comprising the substrate in a variety of ways. For example, a composition or formulation may be injected into a pouf, especially the interior of the pouf. Alternatively, the composition or formulation can be sprayed or coated onto a pouf. Also, a composition or formulation can be sprayed, coated, printed, extruded, or injected into or onto a substrate used to make a personal-care appliance.

Typically soaps, compositions, or other formulations in liquid form will dissipate after 1 or 2 uses. In other words, a substantial portion of the initial quantity of soap, composition, or other formulation associated with the personal-care appliance or substrate will disassociate from the personal-care appliance or substrate during the first use. Disassociation will likely occur through the soap, composition, or formulation dissolving in, or otherwise being carried away by, water during use of the appliance. If the personal-care appliance is used a second time, then that portion of the soap, composition, or other formulation dissipated by the first use is not available for the second use. As stated above, after a few uses, the personal-care appliance. has little or not soap, composition, or other formulation left. If the personal-care appliance is to be adapted for limited use by a user, dissipation of any associated soap, composition, or other formulation provides a signal to the user that the appliance may be disposed of. Manufacturers and/or distributors and/or retailers of the product may explicitly communicate to a purchaser or user that dissipation of the associated soap, composition, or other formulation signals that the appliance may be disposed of.

If the substrate or personal-care appliance is to be adapted for limited use, then the number of times the appliance may be used can be extended in a number of ways. For example, the physical properties of the soap, composition, or other formulation may be altered so that the rate at which the soap or other material dissolves or is carried away is altered. For example, the viscosity of the material may be increased. Alternatively, the soap, composition, or other formulation may be microencapsulated, with the microcapsules making available their contents after some external stimulus is provided (e.g., the microcapsules are broken by the application of an external force as would be present when a user is using the appliance or substrate; or the microcapsule is made using materials known to dissolve in water, with the rate of dissolution of the microcapsules selected so that the availability of the microencapsulated materials during use is extended over the desired number of uses). In another approach, the soap, composition, or other formulation is available in a solid or semi-solid form (as opposed to a liquid), with the rate of dissolution or degradation of the soap selected to for the desired number of uses of the appliance. Soaps, compositions, or formulations may be attached to the substrate or personal-care appliance in some way (for example, solid soaps may be encased in a porous or permeable material such that the solid soap is accessible to water during use of the substrate or personal care appliance). In this way, the substrate or personal-care appliance may be adapted for about 1 to about 5 uses; suitably from about 2 to about 7 uses; or for less than about 10 uses.

Any method for applying or associating a composition or formulation with the substrate and/or appliance may be used, so long as the composition or formulation is adapted, at least in part, to be released from the substrate or appliance during use thereof by a user of the substrate or appliance.

Representative Packages Comprising a Substrate and/or Personal-Care Appliance of the Present Invention

The manufacturer of a personal-care appliance of the present invention (whether a pouf, wash cloth, or other such appliance comprising a substrate of the present invention) may fashion messages, statements, or copy to be transmitted to a purchaser, consumer, or user of said appliance. Such messages, statements, or copy may be fashioned to help facilitate or establish an association in the mind of a user of the appliance between an appliance of the present invention, or use thereof, and one or more mental states, psychological states, or states of well being. The communication, statements, or copy may include various alphanumeric strings, including, for example: relax, peace, energy, energize, sex, sensuality, sensual, spa, spirit, spiritual, clean, fresh, mountain, country, zest, sea, sky, health, hygiene, water, waterfall, moisture, moisturize, derivatives or combinations thereof, or other such states. In one embodiment, the communication, statements, or copy create a mental association in the mind of the consumer between a substrate and/or personal-care appliance of the present invention, and a naturally occurring material, such as a sea sponge. In another embodiment, the communication, statements, or copy create a mental association in the mind of the consumer between a substrate and/or personal-care appliance of the present invention, and a spa or spa-related experience. In another embodiment, the communication, statements, or copy create a mental association in the mind of the consumer between a substrate and/or personal-care appliance of the present invention, and the fact that the substrate and/or personal-care appliance is adapted for a limited use. This latter embodiment may include information on a suggested number of uses, with examples of numbers of uses prior to disposal identified above, and/or information on the benefit of disposal of limited use (e.g., to reduce the chances that various potentially unhealthy microorganisms become associated with the substrate or personal-care appliance).

Alphanumeric strings like those referred to above may be used either alone, adjacent to, or in combination with, other alphanumeric strings. The communication, statements, message, or copy could take the form of (i.e., be embodied in a medium such as) a newspaper advertisement, a television advertisement, a radio or other audio advertisement, items mailed directly to addressees, items emailed to addresses, Internet Web pages or other such postings, free standing inserts, coupons, various promotions (e.g., trade promotions), co-promotions with other companies, copy and the like, boxes and packages containing the product (in this case an appliance of the present invention), and other such forms of disseminating information to consumers or potential consumers. Other exemplary versions of such communications, statements, messages, and/or copy may be found in, for example, U.S. Pat. Nos. 6,612,846 and 6,896,521, both entitled “Method for Displaying Toilet Training Materials and Display Kiosk Using Same”; co-pending U.S. application Ser. No. 10/831,476, entitled “Method of Enunciating a Pre-Recorded Message Related to Toilet Training in Response to a Contact”; co-pending U.S. application Ser. No. 10/956,763, entitled “Method of Manufacturing and Method of Marketing Gender-Specific Absorbent Articles Having Liquid-Handling Properties Tailored to Each Gender”; each of which is incorporated by reference in their entirety in a manner consistent herewith.

It should be noted that when associating statements, copy, messages, or other communications with a package (e.g., by printing text, images, symbols, graphics, color(s), or the like on the package; or by placing printed instructions in the package; or by associating or attaching such instructions, a coupon, or other materials to the package; or the like) containing appliances of the present invention, the materials of construction of said package may be selected to reduce, impede, or eliminate the passage of water or water vapor through at least a portion of the package. Alternatively, the package may be selected to facilitate transmission of water vapor.

As noted above, some embodiments of the present invention comprise a cleaning composition, moisturizing composition, some combination thereof, and the like. Such compositions may contain water. Therefore packages, containers, envelopes, bags, and the like that reduce, minimize, or eliminate the evaporation or transmission of water or water vapor from appliances contained therein may be beneficial. Furthermore, appliances may be individually wrapped in containers, packets, envelopes, bags, or the like that inhibit, reduce, or eliminate the passage or transmission of water or water vapor from appliances contained therein. For purposes of this application, “packages,” “containers,” “envelopes,” “bags,” “packets,” and the like are interchangeable in the sense that they refer to any material adapted to enclose and hold either individual appliances (as in, for example, an individual packet containing a single appliance), or a plurality of appliances (as in a flexible bag made of film containing a plurality of appliances, whether or not each of the individual appliances are enclosed and held in a separate material—such as individual packets).

In other versions of the invention, materials for constructing packages, containers, envelopes, bags, packets, and the like are selected so that the transmission of water or water vapor is facilitated. This may be the case where systematic drying of a personal- care appliance, or substrate, comprising a water-based cleaning composition is desired after the appliance's or substrate's manufacture.

In some embodiments of the present invention, a package will contain not only one or more substrates and/or appliances comprising said substrates, but other personal-care products. In one embodiment, a personal-care appliance of the present invention, such as a pouf, is sold, transferred, distributed, or marketed with other products directed to personal-care, especially products directed to cleaning, moisturizing, or otherwise caring for a user's skin. For example, a substrate or personal-care appliance of the present invention, such as a pouf, can be sold, transferred, distributed, or marketed with a personal-care appliance for moisturizing a user's skin (e.g., hand, foot, forearm, or other locations on a user's body). A co-pending U.S. patent application (U.S. patent application Ser. No. 11/190,597) entitled “Appliance for Delivering a Composition,” filed on 26 Jul. 2005 to K. Close et al., describes such appliances, including socks comprising compositions for moisturizing feet, and gloves comprising compositions for moisturizing hands. This application is hereby incorporated by reference in its entirety in a manner consistent herewith. In another version of the invention, a substrate or personal-care appliance of the present invention, such as a pouf, is sold with a two-sided personal-care appliance, such as a pad having a surface or face primarily for exfoliating skin, and an opposing surface or face primarily for cleaning or moisturizing skin. A co-pending U.S. patent application (U.S. patent application Ser. No. not yet assigned; internal docket number K-C 21998) entitled “Two-Sided Personal Care Appliance for Health, Hygiene, And/Or Environmental Application(s); And Method of Making Said Two-Sided Personal-Care Appliance,” filed on 1 Nov. 2005 to K. Close et al., describes such appliances, including an exfoliating foot buff. This application is hereby incorporated by reference in its entirety in a manner consistent herewith. Other combinations of such personal-care appliances are possible and within the scope of the present invention. It should be noted that such combinations may be marketed and packaged as described in the preceding paragraphs. In one version of the invention, these combinations are marketed in such a way that the design, function, and/or appearance of the individual products making up the combination are related to a common theme. One theme, for example, may be that each product provides a spa-like, or spa-related, treatment or experience for the user of the products. “Spa-like” or “spa-related” relates or refers to a fashionable and/or beneficial treatment or experience analogous to a treatment or experience a guest might receive at a resort, hotel, or other such establishment where a person is refreshed, seeks relaxation, seeks beneficial treatments of his or her skin, hair, muscles, finger nails, toe nails, face, or other parts of the body, and the like.

These and other modifications and variations to the present invention may be practiced by those of ordinary skill in the art, without departing from the spirit and scope of the present invention, which is more particularly set forth in the appended claims. Furthermore, those of ordinary skill in the art will appreciate that the foregoing description is by way of example only, and is not intended to limit the invention so further described in such appended claims.

### EXAMPLES

### Example 1

### Representative Versions of Substrate of Present Invention

Various polymeric materials were obtained and used in preparing an interbonded fibrous layer. Affinity EG 8185 (a metallocene-catalyzed polyethylene material) and Aspun 6806A (a linear, low-density polyethylene), both available from Dow Chemical Company, a business having offices at 2301 Brazosport, Tex., were obtained. SCC 05SAMO6277 (a coral/yellow, polyethylene-based pigment), a polymer obtained from Standridge Color Corporation, a business having offices at 1196 Hightower Trail, Social Circle, Ga., was also obtained. The materials were combined in dry form (as pellets and/or granules) in the following proportions (by weight): 91% Affinity EG 8185; 7% ASPUN 6806A, and 2% SCC 05SAM06277. In some examples (described below), the materials were combined in the following proportions (by weight): 90% Affinity EG 8185; and 10% ASPUN 6806A (i.e., without a pigment). Other proportions may be used; e.g. (by weight): 90% Affinity EG 8185; 7% ASPUN 6806A, and 3% SCC 05SAM06277

The reinforcing strand was made with KRATON-brand GRP 6631, available from KRATON Polymers, a business having offices at 700 Milam, Suite 1300, Houston, Tex. This particular polymer is elastomeric, and is composed of about 85% by weight rubber about 15% by weight wax. No tackifier is present in this particular KRATON-brand material.

To make substrates of the present invention, conveyor belts were obtained from Midwest Industrial Rubber, a business having offices at W6470 Levi Drive, Greenville, Wis. For the prepared substrates, the acquired belts were 15.5 inches wide and 75 inches long (with the belt ends joined together to form an endless belt). The procured belts each had a textured surface. The belts were modified by the manufacturer, in accordance with our specifications, to include die-cut circular holes having a diameter of 0.25 inches. The distance between the centers of the die-cut holes was 0.375 inches in the width dimension of the belt; and 0.375 inches in the length dimension of the belt. The model numbers (with manufacturer's description in brackets) of the acquired belts were MIR 7118 [silicone; endless belt] (this belt used to make the interbonded fibrous layer as described below); MIR 1133 [green RT rough-top; endless belt]; MIR 1111 [white, negative profile; endless belt]; and MIR 1139 [tan, diamond-top; endless belt].

Substrates of the present invention were made using a process like that depicted in FIG. 3. The material for the reinforcing strands, KRATON-brand GRP 6631-1000-09, was added in dry form (as pellets) to a hopper coupled to the extruder. The polymeric material was systematically heated in the body of the extruder, and mixed by the action of the extrusion screw (which mixes and heats the added polymeric material, and propels the heated material to the arrangement of die capillaries), until it was at a temperature of about 395 degrees Fahrenheit. The material was then directed through a bank of capillaries for forming the reinforcing strands (5 holes per inch; 10 inches worth of drilled holes). As stated above, these capillaries were spaced apart in a direction transverse to the direction of travel of the endless belt (i.e., support). The capillaries each had a diameter of 0.050 inches. The extruded KRATON-brand polymer was then directed through, and out of, the capillaries and on to the moving support, with the polymeric reinforcing strands exiting the capillaries at a velocity of about one-tenth of the line speed of the equipment. The tips of the capillaries were about 1.5 inches from the surface of the moving support.

For this particular version of the invention, the reinforcing strands were formed so that they were essentially parallel to one another. Furthermore, the position of the strands were such that the strands could be formed over openings in the support; i.e., the location of the die capillaries relative to the openings on the belt were not selected so that the reinforcing strands would in no event be over an opening. The diameter of the formed reinforcing strands in the finished substrate was about 430 micrometers (these particular strands were substantially circular in cross-section; see Example 5 below for details on measurement). As noted above, the placement of the capillaries relative to openings in the belt may be such that in no event is a reinforcing strand extruded over an opening.

These continuously formed reinforcing strands were then carried by the support to a location directly below the equipment used to form the interbonded fibrous layer (using, in this case, melt-blowing equipment like that depicted in FIG. 3). The location of the tips of the capillaries through which the reinforcing strands were formed was about 2 feet from the location of the tips of the capillaries with which the interbonded fibrous layer was formed. The tips of the capillaries from which the interbonded fibrous layer was formed were about 8 inches from the surface of the moving support. Furthermore, the individual capillaries in the meltblowing die were arranged such that there were 30 holes per inch in a direction transverse to the direction of movement of the support (with a total of 12 inches worth of holes in a direction transverse to the direction of movement of the support). These die capillaries had a diameter of about 0.0145 inch.

Like the polymeric raw material for the reinforcing strands, polymeric ingredients for the interbonded fibrous layer were added to a hopper coupled to an extruder. These polymeric ingredients were then progressively heated until they were blended and had reached a temperature of about 430 degrees Fahrenheit. Polymeric fibers were then formed by directing the molten polymeric material through the capillaries. For this version of the inventive substrate, the primary air temperature of the air used to form the meltblown material was about 520 degrees Fahrenheit. The pressure at which the primary air flow was directed through the meltblowing die was about 28 pounds per square inch (see comments below regarding the location at which this pressure was measured).

Process parameters for each of four codes formed using the preceding polymeric materials are given in Table 1 (“Mb melt temp” gives the temperature, in degrees Fahrenheit, of the meltblown material at a location proximate to its exiting from the capillaries; “Mb Primary Air Temp” gives the temperature, in degrees Fahrenheit, of the heated air that flows around the meltblown material as the material exits the capillaries; “MB Primary Air Pressure” gives the pressure, in pounds per square inch, of the heated air that flows around the meltblown material as the material exits the capillaries—the location at which this pressure was measured is upstream from the bank of capillaries and closer to the compressor source, and therefore higher than the expected 2-3 pounds per square inch of pressure at a location proximate to the location at which the air actually flows around meltblown material exiting the capillaries; “Mb PIH” refers to the pounds [mass] of meltblown material exiting one linear inch of capillaries, in the transverse direction, per hour; “Line Speed” gives the linear velocity, in feet per minute, of the moving support/belt as it moves in a direction transverse to the banks of capillaries through which the interbonded fibrous layer—here, a meltblown material—is formed; “Filament PIH” refers to the pounds [mass] of the filament/reinforcing strand exiting one linear inch of capillaries, in the transverse direction, per hour; “Filament Melt Temp.” gives the temperature of the filament/reinforcing strand at a location proximate to the strands'exiting from the corresponding bank of capillaries; “Filament:Mb Ratio” gives the ratio of the Filament PIH to the Mb PIH; “Basis Weight” gives the weight of the resulting substrate in grams per square meter.)

TABLE 1

| | | Mb | Mb | | | | | | |
|---|---|---|---|---|---|---|---|---|---|
| | Mb | Primary | Primary | | | | Filament | | |
| | melt | Air | Air | | Line | | Melt | | Basis |
| | temp | Temp | Pressure | Mb | Speed | Filament | Temp. | Filament:Mb | Weight |
| Code* | (F) | (F) | (PSI) | PIH | (FPM) | PIH | (F) | Ratio | (gsm) |
| 1 | 430 | 520 | 28 | 0.15 | 5 | 0.15 | 395 | 50:50 | 57 |
| 2 | 430 | 520 | 28 | 0.15 | 7.9 | 0.15 | 395 | 50:50 | 37 |
| 3 | 430 | 520 | 28 | 0.15 | 12.5 | 0.35 | 395 | 70:30 | 40 |

*Codes 1, 2, and 3 were made without a pigment, and included (by weight): 90% Affinity EG 8185; and 10% ASPUN 6806A

As can be seen in Table 1, varying the reinforcing-strand-to-meltblown ratio affects the basis weight of the resulting substrate. The resulting substrates were suitable for use in converting into personal-care appliances, a representative version of which is described in Example 2.

### Example 2

### Representative Personal-Care Appliance

A personal-care appliance, in this case a pouf, was made using a substrate of the present invention (Code 1, prepared as described in Example 1). First a 5-foot length of material was cut from a roll of substrate. Eighty-eight such 5-foot lengths were prepared. Each 5-foot length was then folded in half along the length-wise dimension, with the crease mid-way between the edges (i.e., 5 to 5.5 inches from each edge). The folded-in-half, 5-foot length of material was then heat sealed at its ends (with approximately a ½-inch overlap of the edges). In effect, the 5-foot length of material was converted into an endless tube having approximately a 9 to 10 inch perimeter and a 5-foot length. This tube was then turned inside out so that the heat-sealed edges, with the ½-inch overlap, was inside, rather than outside, the tube. This tube was then placed on two, stainless-steel rods. The bottom edge of the tube was then folded over, and tucked inside, the tube. In effect, an inward-facing, unattached hem was formed at the bottom of the tube. The tube was then systematically gathered by pinching 4-5 inch sections along the 5-foot length dimension and pulling them downward, one on top of the other. In effect, the tube was gathered accordion-like while still mounted on the rods. The top edge of the tube was then folded over, and tucked inside, the tube (creating an inward-facing, unattached hem at the top of the tube). The rods were then pulled apart so that they were approximately 5 inches apart. A 16-inch length of string was then tied around the middle of the gathered, stretched, tube at a point approximately mid-way between the two rods. A square knot was made around the gathered material, with equal lengths of the string extending from this knot. A second square knot was then tied to join these equal lengths of string at their ends (creating a loop of string). The resulting pouf looked similar to the representative pouf depicted in FIGS. 2 and 2A. For this particular version of a personal-care appliance, the appliance was prepared so that the support/belt side of the substrate was oriented outward in the completed personal-care appliance. I.e., the reinforcing strands were oriented outward.

### Example 3

### Performance of Personal-Care Appliance of Present Invention to Comparison Appliance

The personal-care appliance described in Example 2 was tested for its ability to generate foam relative to a conventional pouf made of a netting material (sold by Wal-Mart under the identifier “simply basic”; manufactured by Bradford Soap Mexico Inc.). Basically the personal-care appliances were tested for foam-generation ability by adding a known volume of a cleaning formulation to the appliance. The appliance was then compressed repeatedly in a cell, with the cell positioned over a graduated cylinder. As the appliance was compressed in the cell, lather and liquid would drain from the cell into the cylinder. The volume of this generated lather was then measured.

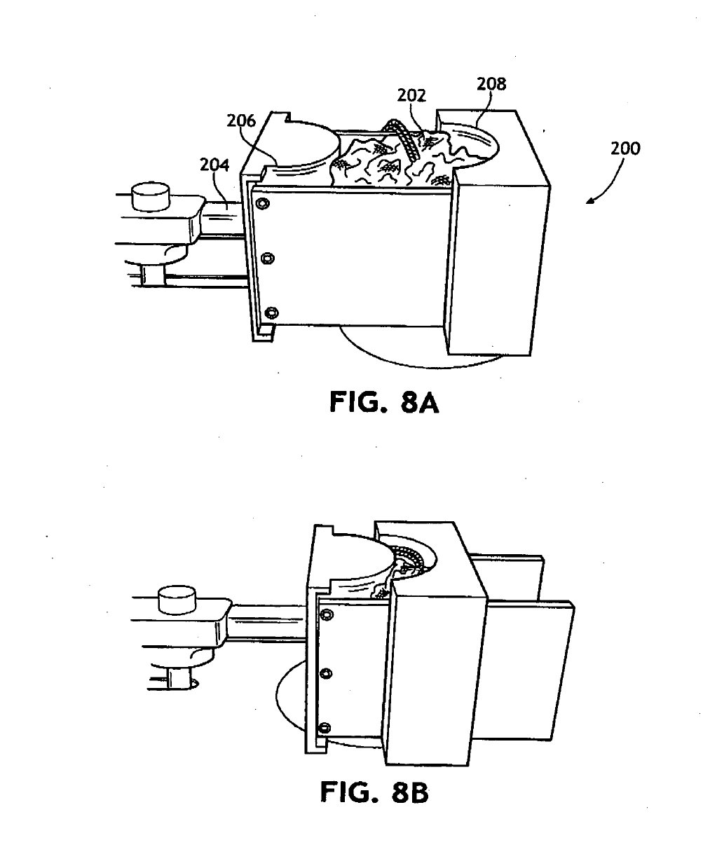

FIGS. 8A and 8B depict the cell 200 into which a personal-care appliance, in this case a pouf 202, is placed. When the test is started, arm 204 moves a portion of the cell having a concave, cylindrical surface 206 against the pouf 202, ultimately compressing the pouf between the concave, cylindirical surface 204 and the convex cylindrical surface 208. FIG. 8B depicts the cell with the personal-care appliance compressed between the aforementioned surface.

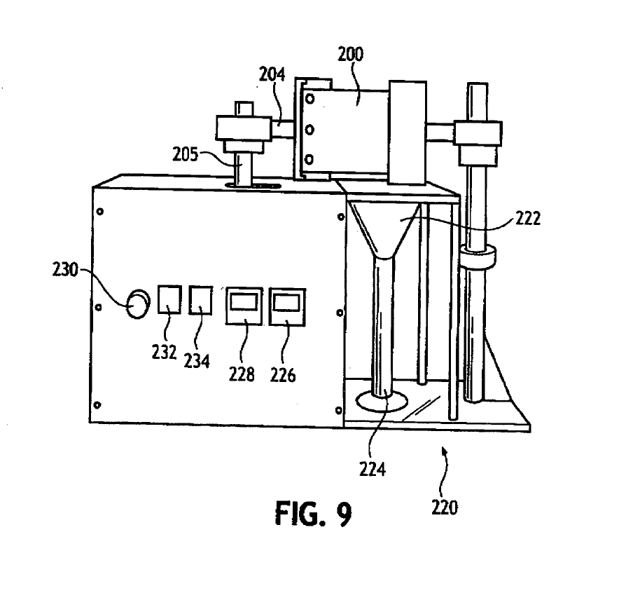

FIG. 9 depicts the apparatus 220 used to conduct the test described in this Example 3. The compression cell 200, generally described in the preceding paragraph and depicted in FIGS. 8A and 8B, is at the top of the apparatus. A drive motor (not shown) is connected to the arm 204 and is used to drive the concave, cylindrical surface of one side of the cell against the personal-care appliance and the convex, cylindrical surface at the opposing side of the cell (see generally description above and FIGS. 8A and 8B). Arm 204 is connected to a vertical support 205, which is connected to a base (not shown) that is able to slide along a rail (also not shown). Various other components (not shown: a programmable logic controller, a drive-motor controller, and a 24-volt DC power supply) are used to power and control the device (see additional detail below). A funnel 222 is positioned immediately below the compression cell to collect and direct liquid and/or foam formed by compression into the graduated cylinder 224. Test parameters that may be selected include: the number of cycle or times the personal-care appliance is to be compressed (selector/display 226 is used to select and display the number of test cycles to be undertaken); the dwell time, or how long the sample is under compression, in seconds (selector/display 228 is used to select and display the dwell time); and the speed at which the compression cycle is accomplished (selector/display 230 is used to select and display the test speed). A test is started when start button 232 is depressed. The compression test is then conducted for the selected number of cycles, each at the selected dwell time and at the selected speed. If the test must be aborted, an operator depresses stop button 234.

For the test results described below, the dwell time was set at 2.3 seconds; the selected number of cycles was 3; and the speed was set at 420 (with these settings corresponding to a cycle time of 2.3 seconds). The pouf to be tested was then weighed. After the pouf was weighed, 7 grams of a soap formulation was added to a location near the center of the pouf. The pouf with the added soap formulation was then placed in a beaker filled with tap water for 5 seconds. A beaker was selected such that there was at least one-half inch clearance between the outer perimeter of the pouf and the interior surface of the side wall of the beaker (the pouf, of course, rested on the bottom of the beaker). Furthermore, the beaker was selected so that, once the pouf was submerged in the water in the beaker, there was at least one-half inch of liquid above the top of the submerged pouf.

After removal of the pouf from the beaker of tap water, the pouf was allowed to drip excess water for 30 seconds. The pouf was then placed in the compression cell described generally in the preceding paragraphs, and the test conducted at the aforementioned test parameters. The volume of foam was then measured (i.e., the volume of foam was measured by determining the volume of foam and liquid together, and then subtracting the volume of liquid that had drained to the bottom of the graduated cylinder). The equipment was then cleaned and the test repeated.

The results of the test are presented in Table 2 below, which gives the volume, in milliliters, of foam generated during the test. As can be seen in the tests, a personal-care appliance of the present invention, in this case a pouf, generated a greater volume of foam than a commercially available pouf made of nylon netting. This was true for each of the three cycles.

TABLE 2

| | | | Commercial Nylon | |
|---|---|---|---|---|
| | Meltblown Pouf | | Pouf | |
| | Average | Std. Dev. | Average | Std. Dev. |
| Foam Generated (ml) Cycle 1 | 27 | 6 | 4 | 1 |
| Foam Generated (ml) Cycle 2 | 48 | 18 | 7 | 2 |
| Foam Generated (ml) Cycle 3 | 48 | 11 | 17 | 11 |

### Example 4

### Comparison between Personal-Care Appliances of Present Invention and Commercially Available Poufs with Respect to Absorbency/Liquid-Holding Ability

The personal-care appliance described in Example 2 was compared to a conventional, commercially available pouf (a pouf made of a plastic netting and sold by Wal-Mart under the identifier “simply basic”; manufactured by Bradford Soap Mexico Inc.) with respect to its liquid-holding capacity. The test was conducted as follows. First the personal-care appliance being tested, in this case a pouf, was weighed using a balance having a 4000-gram capacity. As described generally in Example 4, the pouf was then submerged in a beaker filled with tap water for 5 seconds (the pouf was not injected with a soap formulation prior to being submerged in the tap water). The pouf was then removed from the beaker and allowed to drip excess water for 30 seconds. The weight of the liquid-laden pouf was then determined. The percent liquid-holding capacity (or % absorbent capacity) equated to the weight of the liquid-laden pouf minus the initial weight of the pouf, divided by the initial weight of the pouf.

The absorbent capacities of a personal-care appliance of the present invention (in this case a pouf; identified in the table below as a “Meltblown Pouf”) and a conventional, commercially available pouf were compared. Personal-care appliances of the present invention had greater percent absorbent capacities.

| | | | | % Absorbent |
|---|---|---|---|---|
| | Dry | Wet | Water | Capacity |
| | Weight (g) | Weight (g) | Retention (g) | (g/g) |
| Commercial Pouf 1 | 65 | 105 | 40 | 61 |
| Commercial Pouf 2 | 70 | 105 | 35 | 51 |
| Commercial Pouf 3 | 61 | 99 | 38 | 62 |
| Meltblown Pouf 1 | 29 | 106 | 77 | 262 |
| Meltblown Pouf 2 | 29 | 103 | 73 | 251 |
| Meltblown Pouf 3 | 30 | 118 | 88 | 293 |

| | Average % Absorbent Capacity | Std. Dev. |
|---|---|---|
| Meltblown Pouf | 269 | 22 |
| Commercial Pouf | 58 | 6 |

### Example 5

### Physical Characterization of a Version of a Substrate of the Present Invention

The diameter of meltblown fiber present in an interbonded fibrous layer were determined using scanning-electron-microscope (SEM) images at a magnification of 120×. Six replicate analyses were conducted, with 600-900 individual measurements being taken for each replicate analysis. The mean diameter of the fiber in the interbonded fibrous layer was determined to be 8.9 micrometers (with a standard deviation of 0.6 micrometers). The diameter was determined by measuring the distance along a line perpendicular to the outer perimeter (sides) of a fiber. The distance equated to the distance between the two sides in the two-dimensional image.

The diameter of reinforcing strands was also determined using SEM analysis. Reinforcing strands, in this case, as described above, made from a KRATON-brand material, were stained with osmium tetroxide so that the strands contrasted with the surrounding interbonded fibrous layer (in this case a meltblown material, as discussed generally above). Measurements of diameter were then determined for 35 different strands. The mean diameter of the strands was determined to be 430 micrometers (with a standard deviation of 22 micrometers).

For comparison purposes, the diameter of non-intersecting regions of a lattice-like net in a commercial pouf was determined. The mean diameter of these non-intersecting regions of the lattice-like net was 200 micrometers (with a standard deviation of 20 micrometers).

The size of pores defined by interbonded fiber in the interbonded fiber layer was determined. The equivalent circular diameter was determined for 6 replicate analyses, with each analysis including 300-500 individual measurements. The mean equivalent circular diameter for these pores was 30 micrometers (with a standard deviation of 4 micrometers). Additional detail regarding analyses of equivalent circular diameter is given in U.S. Pat. No. 4,798,603, entitled “Absorbent Article Having a Hydrophobic Transport Layer” and listing Stephen Meyer, et al., as inventors, which is hereby incorporated by reference in its entirety in a manner consistent herewith.

The equivalent circular diameter of approximately 400 openings in eight different fields of views was then measured. The mean diameter measured in this way was 3.2 mm (with a standard deviation of 1.0 mm).

### Example 6

### Cleaning Composition that may be Associated with Substrates and Personal-Care Appliances of the Present Invention

The following ingredients were obtained from the identified supplier, and combined as indicated in the text following the table below.

| | Raw Material | % w/w | Vendor |
|---|---|---|---|
| 1 | Water, USP | 36.40 | |
| 2 | Plantapon 611L | 20.00 | Cognis |
| | | | Ambler, PA |
| 3 | Glycerin, 99.5% USP | 10.00 | Ruger Chemicals |
| | | | Linden |
| 4 | Velvetex CDC | 5.00 | Cognis |
| 5 | Plantapon ACG 50 | 5.00 | Cognis |
| 6 | 1,3, Butylene Glycol | 5.00 | Ruger Chemicals |
| 7 | Lamesoft PO 65 | 3.00 | Cognis |
| 8 | Cetiol HE | 2.00 | Cognis |
| 9 | Polyquart 701 NA | 2.00 | Cognis |
| 10 | Elestab FL-15 | 2.00 | Cognis |
| 11 | Actiphyte of Jojoba Meal BG50P | 2.00 | Active Organics |
| | | | Lewisville, TX |
| 12 | Actiphyte of Avocado BG50P | 2.00 | Active Organics |
| 13 | Actiphyte of Aloe Vera 10 fold | 2.00 | Active Organics |
| | BG50P | | |
| 14 | Tinoderm A | 1.00 | Ciba Specialty |
| | | | Chemicals |
| | | | High Point, NC |
| 15 | dl-Panthenol, USP | 1.00 | Ruger Chemicals |
| 16 | Fragrance | 1.00 | |
| 17 | Citric Acid, USP | 0.40 | Sigma |
| | | | St. Louis, MO |
| 18 | Edetate Disodium Dihydrate, USP | 0.10 | Sigma |
| 19 | Vitamin E Acetate, USP | 0.10 | Ruger Chemicals |
| | TOTAL | 100.00 | |

The recited proportions of ingredients 3 through and including 14, and 19, were added to the recited weight proportion of water (minus those amounts of water used in separate steps, as described below) and mixed together in a Lightnin Labmaster mixer LIU10F (135 Mt. Read Blvd., Rochester, N.Y.). Three percent of the formula weight of water was then heated to a temperature between about 45 and 50 degrees Celsius. To the heated water was added edentate disodium in the proportion recited above, which was then mixed. To this mixture was then added panthenol in the proportion recited above, which was then mixed further. This combination of heated water, edentate disodium, and panthenol was then added to the aqueous mixture having 97% of the recited formula weight of water. Plantapon 611L was added to this mixture, which was further mixed to disperse this newly added ingredient. A 50% solution of citric acid was then prepared (utilizing 0.8% of the formula weight of water). The pH was then adjusted by adding the citric acid solution, as needed, to obtain a pH of between 5.5 and 6.5. Finally, fragrance was added, with the cleaning composition mixed to obtain a homogeneous dispersion.

The cleaning composition was then applied to a personal-care appliance of the present invention, in this case by using a syringe to inject about 7 grams into the center of a pouf made using a substrate comprising an interbonded fibrous layer comprising shaped discontinuities, to which were attached reinforcing strands (code 1 described in Example 1 above). Use of the personal-care appliance treated with the cleaning composition described above resulted in the formation of lather useful for cleaning and/or treating the skin.

### Example 7

### Cleaning Composition that may be Associated with Substrates and Personal-Care Appliances of the Present Invention

The following ingredients were obtained from the identified supplier, and combined as indicated in the text following the table below.

| | Raw Material | % w/w | Vendor |
|---|---|---|---|
| 1 | Water, USP | 30.40 | |
| 2 | Plantapon 611L | 20.00 | Cognis |
| 3 | Glycerin, 99.5% USP | 10.00 | Ruger Chemicals |
| 4 | Velvetex CDC | 5.00 | Cognis |
| 5 | Plantapon ACG 50 | 5.00 | Cognis |
| 6 | 1,3, Butylene Glycol | 5.00 | Ruger Chemicals |
| 7 | Lamesoft PO 65 | 3.00 | Cognis |
| 8 | Cetiol HE | 2.00 | Cognis |
| 9 | Polyquart 701 NA | 2.00 | Cognis |
| 10 | Elestab FL-15 | 2.00 | Cognis |
| 11 | Actiphyte of Jojoba Meal BG50P | 2.00 | Active Organics |
| 12 | Actiphyte of Avocado BG50P | 2.00 | Active Organics |
| 13 | Actiphyte of Aloe Vera 10 fold | 2.00 | Active Organics |
| | BG50P | | |
| 14 | Tinoderm A | 1.00 | Ciba Specialty |
| | | | Chemicals |
| 15 | dl-Panthenol, USP | 1.00 | Ruger Chemicals |
| 16 | Fragrance | 1.00 | |
| 17 | Citric Acid, USP | 0.40 | Sigma |
| 18 | Edetate Disodium Dihydrate, USP | 0.10 | Sigma |
| 19 | Vitamin E Acetate, USP | 0.10 | Ruger Chemicals |
| 20 | Jojoba Spheres 20 | 4.0 | Desert Whale |
| | | | Jojoba Co., |
| | | | Tucson, AZ |
| 21 | Microscrub 20 | 2.0 | Presperse Inc., |
| | | | Somerset, NJ |
| | TOTAL | 100.00 | |

The recited proportions of ingredients 3 through and including 14, and 19, were added to recited formula weight of water (minus those amounts of water used in separate steps, as described below) and mixed together in a Lightnin Labmaster mixer LIU10F (135 Mt. Read Blvd., Rochester, N.Y.). Three percent of the formula weight of water was then heated to a temperature between about 45 and 50 degrees Celsius. To the heated water was added edentate disodium in the proportion recited above, which was then mixed to dissolve the added ingredient. To this heated mixture was then added Panthenol in the proportion recited above, which was then mixed to dissolve this second ingredient. This combination of heated water, edentate disodium, and panthenol was then added to the previously prepared aqueous mixture. Plantapon 611L was added to this mixture, which was further mixed to disperse this newly added ingredient. Ingredients 20 and 21 were added to the combination to aid in exfoliating and/or stimulating skin during use of the personal-care appliance. A 50% solution of citric acid was then prepared (utilizing 0.8% of the formula weight of water). The pH was then adjusted by adding the citric acid solution, as needed, to obtain a pH of between 5.5 and 6.5. Finally, fragrance was added, with the cleaning composition mixed to obtain a homogeneous dispersion.

The cleaning composition was then applied to a personal-care appliance of the present invention, in this case by using a syringe to inject about 7 grams into the center of a pouf made using a substrate comprising an interbonded fibrous layer comprising shape discontinuities, to which were attached reinforcing strands (code 1 described in Example 1 above). Use of the personal-care appliance treated with the cleaning composition described above resulted in the formation of a lather useful for cleaning and/or treating the skin.

We claim:

1. A substrate comprising:
an interbonded fibrous layer comprising interbonded fibers having a mean first diameter, and shaped discontinuities in said interbonded fibrous layer; and
a plurality of spaced-apart, reinforcing strands having a mean second diameter and attached to at least a portion of the interbonded fibrous layer.

2. The substrate of claim 1 wherein each of the spaced-apart, reinforcing strands are generally parallel to one another.

3. The substrate of claim 2 wherein the shaped discontinuities are generally circular.

4. The substrate of claim 3 wherein the shaped discontinuities are contiguous with projections emanating from the surface of the interbonded fibrous layer.

5. A personal-care appliance comprising the substrate of claim 1.

6. The personal-care appliance of claim 5 comprising a cleaning composition.

7. The personal-care appliance of claim 5 wherein the appliance is a pouf.

8. The pouf of claim 7 comprising a cleaning composition.

9. The substrate of claim 3 wherein: the interbonded fibers of the interbonded fibrous layer define pores having a mean pore diameter; the generally circular shaped discontinuities define a mean diameter; and the mean diameter of the circular shaped discontinuities is larger than the mean pore diameter of the pores defined by the interbonded fibrous layer.

10. The substrate of claim 9 where in the ratio of the mean diameter of the circular shaped discontinuities to the mean pore diameter of the pores defined by the interbonded fibrous layer is at least about 10 to 1.

11. The substrate of claim 9 where in the ratio of the mean diameter of the circular shaped discontinuities to the mean pore diameter of the pores defined by the interbonded fibrous layer is at least about 100 to 1.

12. The substrate of claim 1 wherein the mean first diameter is smaller than the mean second diameter.

13. The substrate of claim 1 wherein the ratio of the mean first diameter to the mean second diameter is at least about 10 to 1.

14. The substrate of claim 1 wherein the ratio of the mean first diameter to the mean second diameter is at least about 100 to 1.

15. The substrate of claim 1 wherein the interbonded fibrous layer comprises fiber that is elastomeric.

16. The substrate of claim 1 wherein the reinforcing strands are adapted to contact the skin of a user of the substrate during use of the substrate.

17. The personal-care appliance of claim 5 wherein the reinforcing strands are adapted to contact the skin of a user of the appliance during use of the appliance.

18. A method of making a substrate, the method comprising the steps of:
(a) forming reinforcing filaments on a moving support having openings;
(b) forming an interbonded fibrous layer on a moving support having openings;
(c) bonding at least a portion of said reinforcing filaments to said interbonded fibrous layer;
(d) moving at least some portion of the interbonded fibrous layer proximate to at least some of said openings into at least some of said openings thereby forming shaped discontinuities in said interbonded fibrous layer.

19. The method of claim 18 wherein substantially no waste is formed during the making of the substrate.

20. The method of claim 18 wherein at least some portion of the reinforcing strands proximate to at least some of said openings move into at least some of said openings.

21. The method of claim 20 wherein at least some portions of the reinforcing strands separate.

22. A method of making a personal-care appliance, the method comprising the steps of:
providing a substrate formed by the method of claim 18;
converting the substrate into a personal-care appliance.

23. A package, the package comprising:
a container; and
one or more personal-care appliances of claim 5 contained in said container.

24. The package of claim 23 wherein the container is impermeable to water and water vapor.

25. The package of claim 24 wherein each personal-care appliance is further contained in a separate envelope, wherein each envelope is impermeable to water and water vapor.

26. The package of claim 23 further comprising a statement on or in the package, said statement relating the appliance to spa-related activities or experiences.

27. The package of claim 23 further comprising a statement on or in the package referring to the appliance being adapted for limited use by a user of the appliance.

28. The package of claim 23 further comprising one or more of the following alphanumeric strings: relax, peace, energy, energize, sex, sensuality, sensual, spa, spirit, spiritual, clean, fresh, mountain, country, zest, sea, sky, health, hygiene, water, waterfall, moisture, moisturize, or some combination thereof.

29. The package of claim 23 further comprising a personal-care appliance for moisturizing skin, a two-sided personal-care appliance for exfoliating skin, or both.

30. The package of claim 23 wherein the container is permeable to water and water vapor to facilitate transmission of water or water vapor from personal-care appliances contained therein.

31. The package of claim 30 wherein each personal-care appliance is further contained in a separate envelope, wherein each envelope is permeable to water and water vapor to facilitate transmission of water or water vapor from personal-care appliances contained therein.
